(19) United States
(12) Patent Application Publication
(10) Pub. No.: US 2026/0183463 A1
(43) Pub. Date: Jul. 2, 2026

(54) ROTARY PUMP WITH ELECTRICALLY HEATED DETACHABLE PUMP HEAD FOR ECLS APPLICATIONS

(71) Applicant: Maquet Cardiopulmonary GmbH, Rastatt (DE)

(72) Inventors: Verena BINZ, Gaggenau (DE); Janpeter HÖFFMANN, Bad Zwischenahn (DE); Cyril REITER, Roeschwoog (FR); Andreas KORELL, Balingen (DE); Mathias NAKEL, Sonnenbühl (DE); Rudolf KOBER, Baden-Baden (DE)

(21) Appl. No.: 19/548,826

(22) Filed: Feb. 24, 2026

Related U.S. Application Data
(63) Continuation of application No. PCT/EP2024/074001, filed on Aug. 28, 2024.
(60) Provisional application No. 63/579,075, filed on Aug. 28, 2023.

(51) Int. Cl. A61M 1/36; A61M 60/113; A61M 60/216; A61M 60/38; A61M 60/804

(52) U.S. Cl. CPC A61M 1/369; A61M 60/113; A61M 60/216; A61M 60/38; A61M 60/804; A61M 2205/366; A61M 2205/368

(57) ABSTRACT

Devices, systems, and methods for warming the patient during an ECLS procedure. Depending on the specific need (heart and lung support or pure heart support) the patient may be warmed via a heat exchanger in which the patient bloods comes into close—indirect—contact with a warming fluid or, in a pure Heart Assist procedure, by directly warming the blood of the patient. The proposed solution to both warming methods described above is a centrifugal pump with an integrated heating which has a detachable pump head. The invention allows for a smaller design, simpler handling, using a sterile warming fluid with no air contact, turbulence enhanced heat transfer and includes inherent safety aspects for ECLS patients.

### CROSS-REFERENCE TO RELATED APPLICATIONS

This application is a continuation of International Application No. PCT/EP2024/074001, filed Aug. 28, 2024, which claims the benefit of U.S. Provisional Application No. 63/579,075, filed Aug. 28, 2023. Each of the above-referenced patent applications is incorporated by reference in its entirety.

### BACKGROUND OF THE DISCLOSURE

### Field of the Disclosure

The present disclosure relates to pumps with integrated heating elements for heating a pumped fluid, more particularly to pumps with integrated heating elements consisting of 2 separable components, one first component the fluid goes through and a second component having no contact to the pumped fluid containing the pump drive.

### Description of Related Art

Extracorporeal Life Support (ECLS) is a technique of providing prolonged cardiac and/or respiratory support to an individual whose own heart and/or lungs are/is unable to sustain life. The most often used ECLS approach is the ECMO procedure, in which the patient is connected to an extra corporeal circulation system including a blood pump and oxygenator to provide blood perfusion and gas exchange. In ECLS cases where the lungs of the patient are completely well and only heart support is needed the Heart Assist system includes only a blood pump to provide blood perfusion but no oxygenator.

During an ECMO or Heart Assist procedure, the patient's core body temperature often drops, for example, as a result of the patient's blood being circulated externally of the patient's body. In some cases, the patient's body core temperature may be low even before the ECLS procedure, such as if the patient is hypothermic. As such, the blood may be heated prior to being re-introduced into the patient to maintain a suitable body core temperature.

One method of heating the blood is via a heat exchanger in which a warming fluid is brought into indirect contact with the blood to raise the blood temperature. The heat exchanger could be a stand alone device (FIG. 13) or built in the oxygenator (FIG. 12). The heated blood is then returned to the patient.

The warming fluid itself can be heated using a variety of methods, such as providing a heating element in a flow path of the warming fluid. However, existing methods of heating the warming fluid have several deficiencies, such as inconsistent heating of the warming fluid, slow response time, and generally large and cumbersome size. In addition, existing heating devices are not inherenetly (or even optionally) sterile. As such, the warming fluid may become contaminated, meaning the heating devices need to be disinfected at regular intervals to ensure safe operation. This leads to downtime and increased workload of the devices. As a result of these disadvantages and deficiencies of the prior art, there exists a need for improved devices, systems, and methods for warming the patient during an ECLS procedure that are efficient and sterile.

Another method of heating the blood is heating the blood directly in the pump without using a heat exchanger (FIG. 14). For this procedure no prior art is known and therefore this new method together with an apparatus using this new method is described in this invention disclosure.

### SUMMARY OF THE DISCLOSURE

The present disclosure addresses the aforementioned disadvantages and deficiencies of the prior art by providing devices, systems, and methods for efficiently warming a patient while maintaining sterility. In particular, the present disclosure is directed to devices, systems, and methods for warming the patient during an ECLS procedure. Depending on the specific need (heart and lung support or pure heart support) the patient may be warmed via a heat exchanger in which the patient bloods comes into close-indirect-contact with a warming fluid or, in a pure Heart Assist procedure, by directly warming the blood of the patient.

The proposed solution to both warming methods described above is a centrifugal pump with an integrated heating which has a detachable pump head.

The invention has the following advantages:
The integration of pump and heating allows for a smaller design. Firstly, this is advantageous for the transport of a patient being on ECLS. Secondly, because the priming volume of heater circuit can be reduced which allows a faster response time after parameter changes and by that a more dynamic heating performance.
The detachability of pump head allows for a simpler handling as the pump head can be exchanged during use. Also repeated usage of the heating pump is easily possible by removing the used pump head and replace it with a new one.
Using a detachable and disposable pump head allows for a sterile disposable circuit which means having a sterile warming fluid with no air contact. Thus, there is no possibility of bacteria growth inside the warming fluid.
The combination of heating and pumping in the same chamber allows for a turbulence enhanced heat transfer from the heating parts to the fluid to be heated. Also, mixing of the heated fluid with not yet heated fluid is improved by micro and macro turbulences.
The invention has also a safety aspect for ECLS patients. As the heat transfer rate is dependent on the rotational speed of the pump, the risk of overheating the patient can be controlled by the rotational speed. In the extreme, when the pump is stopped no heat is transferred anymore. Moreover, as an additional safety feature, this invention reduces the risk of the occurrence of “hot spots”.

Different embodiments of the centrifugal pump with an integrated heating which has a detachable pump head are proposed.

The heating could be inductive or resistive. The inductive heating could be based on a alternating magnetic field or on stationary magnetic field. For the inductive heating based on an alternating magnetic field as well as for the inductive heating based on an a stationary magnetic field different embodiments of the inductor and/or conductor are discussed.

Moreover, an embodiment of the centrifugal pump with an integrated heating which has a detachable pump head is proposed in which the inductive heating is combined with an electromagnetic pump drive.

In one non-limiting example or aspect of the present disclosure, a heating fluid pump assembly for a fluid flow system may include a first component comprising an impeller configured to circulate a fluid through an impeller chamber; a second component comprising the drive of the impeller; and an inductive heating element, the inductive heating element comprising a conductor which is part of the first component, and an inductor which is part of second component, fluidly isolated from the conductor, wherein the first component is detachable from the second component, wherein the inductor is configured to generate a magnetic field to induce eddy currents in the conductor, wherein the heating element is configured to induce eddy currents in the conductor in order to heat the conductor, and wherein the conductor is configured to contact the fluid as the fluid is circulated by the impeller.

In one non-limiting example or aspect of the present disclosure, the inductive heating element may include an inductor coil. A controller may be provided and configured to supply electrical power to the inductor coil to generate the magnetic field. The inductor coil may include one or more windings. The one or more windings may be arranged in a single layer. The one or more windings may be arranged in multiple layers. The inductor coil may be a printed circuit board. The conductor may be rotationally coupled to the impeller. The impeller as a whole may be made of conductive material so that the impeller is the conductor at the same time. The conductive impeller may include an arrangement to increase the surface resulting in an increased heat exchange area and induces micro-turbulence in the fluid. The conductor may be configured as a conductive layer applied to an inner surface of the impeller chamber. A controller may be provided and configured to supply electrical power to the conductive layer. The conductor may be configured as a stationary heating component affixed to the impeller chamber.

In one non-limiting example or aspect of the present disclosure, a heating fluid pump assembly for a fluid flow system may include a first component comprising an impeller configured to circulate a fluid through an impeller chamber; a second component comprising a drive of the impeller; and an inductive heating element, the inductive heating element comprising a conductor which is part of the first component, and a magnet which is part of second component, fluidly isolated from the conductor, wherein the first component is detachable from the second component, wherein the magnet is configured to generate a magnetic field to induce eddy currents in the conductor, wherein the heating element is configured to induce eddy currents in the conductor in order to heat the conductor, and wherein the conductor is configured to contact the fluid as the fluid is circulated by the impeller.

In one non-limiting example or aspect of the present disclosure, the conductor may include a lug extending between sidewalls of the magnet. The magnet may be a permanent magnet.

In one non-limiting example or aspect of the present disclosure, a heating fluid pump assembly for a fluid flow system may include a first component comprising an impeller configured to circulate a fluid through an impeller chamber; a second component comprising a drive of the impeller; and an electric heating element, the electric heating element comprising an electrical resistor which is part of the first component, and an electrical energy source which is part of second component, wherein the first component is detachable from the second component, wherein the electrical resistor is electrically connected to the electrical energy source, wherein the electric heating is configured to heat the electrical resistor, and wherein the electrical resistor is configured to contact the fluid as the fluid is circulated by the impeller.

In one non-limiting example or aspect of the present disclosure, the impeller as a whole may be made of a electrical resistor material so that the impeller itself is the electrical resistor. The conductive impeller may have an arrangement to increase the surfaces resulting in an increased heat exchange area and induces micro-turbulence in the fluid. The electrical resistor may include a conductive layer applied to the impeller. The electrical resistor may be at least a portion of the impeller. The electrical resistor may include a conductive layer applied to an inner surface of the impeller chamber. The electrical resistor may be electrically connected to a shaft for rotating the impeller.

In one non-limiting example or aspect of the present disclosure, a heating fluid pump assembly for a fluid flow system may include a first component comprising an impeller configured to circulate a fluid through an impeller chamber, a second component comprising the electromagnetic drive of the impeller; and an inductive heating element, the inductive heating element comprising a conductor which is part of the first component, and an inductor which is part of second component, wherein the first component is detachable from the second component, wherein the heating element inductor is configured to generate a magnetic field to induce eddy currents in the conductor, wherein the heating element is configured to induce eddy currents in the conductor in order to heat the conductor, wherein the conductor is configured to contact the fluid as the fluid is circulated by the impeller, wherein the impeller comprises one or more magnets to interacte with the drive of the impeller, and wherein the electromagnetic drive of the impeller is configured to generate a rotating magnetic field to rotate the impeller via induction.

In one non-limiting example or aspect of the present disclosure, the electromagnetic drive may be a drive coil. The heating element inductor may serve at the same time as the inductive coil of the electromagnetic drive. The heating element inductor and the inductive coil of the electromagnetic drive may be separate coils. The heating fluid pump assembly may include a controller configured to supply electrical power to the heating element inductor coil to generate the magnetic field for heating. The heating fluid pump assembly may include a controller configured to supply electrical power to the drive coil to generate a rotary magnetic field to rotate the impeller.

In one non-limiting example or aspect of the present disclosure, a heating fluid pump assembly for a fluid flow system, wherein the first component comprising an impeller and an impeller chamber is connected via tubes to a heat exchanger to constitute a warming fluid circuit and wherein the warming fluid circuit is configured to circulate a warming fluid through the impeller chamber and through the heat exchanger. The warming fluid circuit may be prefilled with a sterile warming fluid and wherein the sterile warming fluid circulates through the heat exchanger.

In one non-limiting example or aspect of the present disclosure, the heating fluid pump assembly for a fluid flow system for ECMO applications, wherein an initial warming fluid circuit comprising of the said first component comprising an impeller and an impeller chamber and of a conduit connecting input and output of the impeller chamber is prefilled with a sterile warming fluid and wherein the initial warming fluid circuit is connected under sterile conditions to the heat exchanger integrated in an oxygenator so that during the ECMO case sterile warming fluid circulates through the heat exchanger.

In one non-limiting example or aspect of the present disclosure, The heating fluid pump assembly for a fluid flow system, comprising at least one of an ECMO machine, a heart-lung machine, and a dialysis machine, wherein the circulating fluid is blood, and wherein the blood is circulated and heated in a circuit comprising the extracorporeal part of the circuit, the circulatory system of the patient and at least one inflow and one outflow arrangement connecting the extracorporeal part of the circuit to the circulatory system of the patient, wherein the extracorporeal part of the circuit comprises the the first component comprising an impeller and an impeller chamber, an oxygenator, and conduits connecting the first component to the oxygenator and to the inflow and outflow arrangement.

In one non-limiting example or aspect of the present disclosure, the heating fluid pump assembly for a fluid flow system, comprising at least one of an ECLS machine, a heart-lung machine, and a dialysis machine, wherein the circulating fluid is blood, and wherein the blood is circulated and heated in a circuit comprising the extracorporeal part of the circuit, the circulatory system of the patient and at least one inflow and one outflow arrangement connecting the extracorporeal part of the circuit to the circulatory system of the patient, wherein the extracorporeal part of the circuit comprises the first component comprising an impeller and an impeller chamber and conduits connecting the first component to the inflow and outflow arrangement.

The present invention is also disclosed in the following clauses:
Clause 1: A heating fluid pump assembly for a fluid flow system, the heating fluid pump assembly comprising: a first component comprising an impeller configured to circulate a fluid through an impeller chamber; a second component comprising the drive of the impeller; and an inductive heating element, the inductive heating element comprising a conductor which is part of the first component, and an inductor which is part of second component, fluidly isolated from the conductor, wherein the first component is detachable from the second component, wherein the inductor is configured to generate a magnetic field to induce eddy currents in the conductor, wherein the heating element is configured to induce eddy currents in the conductor in order to heat the conductor, and wherein the conductor is configured to contact the fluid as the fluid is circulated by the impeller.
Clause 2: The heating fluid pump assembly of Clause 1, wherein the inductive heating element comprises an inductor coil.
Clause 3: The heating fluid pump assembly of Clause 2, further comprising a controller configured to supply electrical power to the inductor coil to generate the magnetic field.
Clause 4: The heating fluid pump assembly of Clause 2, wherein the inductor coil comprises one or more windings.
Clause 5: The heating fluid pump assembly of Clause 4, wherein the one or more windings are arranged in a single layer.
Clause 6: The heating fluid pump assembly of Clause 4, wherein the one or more windings are arranged in multiple layers.
Clause 7: The heating fluid pump assembly of Clause 4, wherein the inductor coil is a printed circuit board.
Clause 8: The heating fluid pump assembly of any of Clauses 1-7, wherein the conductor is rotationally coupled to the impeller.
Clause 9: The heating fluid pump assembly of any of Clauses 1-8, wherein the impeller as a whole is made of conductive material so that the impeller is the conductor at the same time.
Clause 10: The heating fluid pump assembly of Clause 9, wherein the conductive impeller includes an arrangement to increase the surface resulting in an increased heat exchange area and induces micro-turbulence in the fluid.
Clause 11: The heating fluid pump assembly of any of Clauses 1-10, wherein the conductor is configured as a conductive layer applied to an inner surface of the impeller chamber.
Clause 12: The heating fluid pump assembly of Clause 11, further comprising a controller configured to supply electrical power to the conductive layer.
Clause 13: The heating fluid pump assembly of any of Clauses 1-12, wherein the conductor is configured as a stationary heating component affixed to the impeller chamber.
Clause 14: A heating fluid pump assembly for a fluid flow system, the heating fluid pump assembly comprising a first component comprising an impeller configured to circulate a fluid through an impeller chamber; a second component comprising a drive of the impeller; and an inductive heating element, the inductive heating element comprising a conductor which is part of the first component, and a magnet which is part of second component, fluidly isolated from the conductor, wherein the first component is detachable from the second component, wherein the magnet is configured to generate a magnetic field to induce eddy currents in the conductor, wherein the heating element is configured to induce eddy currents in the conductor in order to heat the conductor, and wherein the conductor is configured to contact the fluid as the fluid is circulated by the impeller.
Clause 15: The heating fluid pump assembly of Clause 14, wherein the conductor comprises a lug extending between sidewalls of the magnet.
Clause 16: The heating fluid pump assembly of Clause 15, wherein the magnet is a permanent magnet.
Clause 17: A heating fluid pump assembly for a fluid flow system, the heating fluid pump assembly comprising: a first component comprising an impeller configured to circulate a fluid through an impeller chamber; a second component comprising a drive of the impeller; and an electric heating element, the electric heating element comprising an electrical resistor which is part of the first component, and an electrical energy source which is part of second component, wherein the first component is detachable from the second component, wherein the electrical resistor is electrically connected to the electrical energy source, wherein the electric heating is configured to heat the electrical resistor, and wherein the electrical resistor is configured to contact the fluid as the fluid is circulated by the impeller.
Clause 18: The heating fluid pump assembly of Clause 17, wherein the impeller as a whole is made of a electrical resistor material so that the impeller itself is the electrical resistor.
Clause 19: The heating fluid pump assembly of Clause 18, wherein the conductive impeller has an arrangement to increase the surfaces resulting in an increased heat exchange area and induces micro-turbulence in the fluid.
Clause 20: The heating fluid pump assembly of any of Clasues 17-19, wherein the electrical resistor comprises a conductive layer applied to the impeller.
Clause 21: The heating fluid pump assembly of any of Clauses 17-20, wherein the electrical resistor is at least a portion of the impeller.
Clauses 22: The heating fluid pump assembly of any of Clauses 17-21, wherein the electrical resistor comprises a conductive layer applied to an inner surface of the impeller chamber.
Clause 23: The heating fluid assembly of any of Clauses 17-22, wherein the electrical resistor is electrically connected to a shaft for rotating the impeller.
Clause 24: A heating fluid pump assembly for a fluid flow system, the heating fluid pump assembly comprising: a first component comprising an impeller configured to circulate a fluid through an impeller chamber, a second component comprising the electromagnetic drive of the impeller; and an inductive heating element, the inductive heating element comprising a conductor which is part of the first component, and an inductor which is part of second component, wherein the first component is detachable from the second component, wherein the heating element inductor is configured to generate a magnetic field to induce eddy currents in the conductor, wherein the heating element is configured to induce eddy currents in the conductor in order to heat the conductor, wherein the conductor is configured to contact the fluid as the fluid is circulated by the impeller, wherein the impeller comprises one or more magnets to interacte with the drive of the impeller, and wherein the electromagnetic drive of the impeller is configured to generate a rotating magnetic field to rotate the impeller via induction.
Clause 25: The heating fluid pump assembly of Clause 24, wherein the electromagnetic drive comprises a drive coil.
Clause 26: The heating fluid pump assembly of Clause 25, wherein the heating element inductor serves at the same time as the inductive coil of the electromagnetic drive.
Clause 27: The heating fluid pump assembly of Clause 25, wherein the heating element inductor and the inductive coil of the electromagnetic drive are separate coils.
Clause 28: The heating fluid pump assembly of Clause 25, wherein the heating fluid pump assembly further comprises a controller configured to supply electrical power to the heating element inductor coil to generate the magnetic field for heating.
Clause 29: The heating fluid pump assembly of Clause 25, wherein the heating fluid pump assembly further comprises a controller configured to supply electrical power to the drive coil to generate a rotary magnetic field to rotate the impeller.
Clause 30: A heating fluid pump assembly for a fluid flow system according to Clause 1, 14, 17 or 24, wherein the first component comprising an impeller and an impeller chamber is connected via tubes to a heat exchanger to constitute a warming fluid circuit and wherein the warming fluid circuit is configured to circulate a warming fluid through the impeller chamber and through the heat exchanger.
Clause 31: The heating fluid pump assembly for a fluid flow system of Clause 30 wherein the warming fluid circuit is prefilled with a sterile warming fluid and wherein the sterile warming fluid circulates through the heat exchanger.
Clause 32: The heating fluid pump assembly for a fluid flow system of Clause 30 for ECMO applications, wherein an initial warming fluid circuit comprising of the said first component comprising an impeller and an impeller chamber and of a conduit connecting input and output of the impeller chamber is prefilled with a sterile warming fluid and wherein the initial warming fluid circuit is connected under sterile conditions to the heat exchanger integrated in an oxygenator so that during the ECMO case sterile warming fluid circulates through the heat exchanger.
Clause 33: The heating fluid pump assembly for a fluid flow system according to Clause 1, 14, 17 or 24, comprising at least one of an ECMO machine, a heart-lung machine, and a dialysis machine, wherein the circulating fluid is blood, and wherein the blood is circulated and heated in a circuit comprising the extracorporeal part of the circuit, the circulatory system of the patient and at least one inflow and one outflow arrangement connecting the extracorporeal part of the circuit to the circulatory system of the patient, wherein the extracorporeal part of the circuit comprises the the first component comprising an impeller and an impeller chamber, an oxygenator, and conduits connecting the first component to the oxygenator and to the inflow and outflow arrangement.
Clause 34: The heating fluid pump assembly for a fluid flow system according to Clause 1, 14, 17 or 24, comprising at least one of an ECLS machine, a heart-lung machine, and a dialysis machine, wherein the circulating fluid is blood, and wherein the blood is circulated and heated in a circuit comprising the extracorporeal part of the circuit, the circulatory system of the patient and at least one inflow and one outflow arrangement connecting the extracorporeal part of the circuit to the circulatory system of the patient, wherein the extracorporeal part of the circuit comprises the first component comprising an impeller and an impeller chamber and conduits connecting the first component to the inflow and outflow arrangement.

Further details and advantages of the various non-limiting examples described in detail herein will become clear upon reviewing the following detailed description of the various non-limiting examples in conjunction with the accompanying drawing figures.

### BRIEF DESCRIPTION OF THE DRAWINGS

FIG. 1 is a schematic diagram of an extracorporeal circulation system in accordance with an embodiment of the present disclosure;

FIG. 2 is a schematic diagram of an extracorporeal circulation system in accordance with an embodiment of the present disclosure;

FIG. 3 is a cross-sectional view of a fluid heating pump assembly, in accordance with an embodiment of the present disclosure;

FIG. 4 is a schematic view of a fluid heating pump assembly, in accordance with an embodiment of the present disclosure;

FIG. 5 is a perspective cross-sectional view of a fluid heating pump assembly, in accordance with an embodiment of the present disclosure;

FIG. 6 is a side cross-sectional view of the fluid heating pump assembly of FIG. 5;

FIG. 7 is a perspective view of the fluid heating pump assembly of FIG. 5, with the impeller chamber and housing thereof not shown for clarity;

FIG. 8 is a cross-sectional view of a fluid heating pump assembly, in accordance with an embodiment of the present disclosure;

FIG. 9 is a cross-sectional view of a fluid heating pump assembly, in accordance with an embodiment of the present disclosure;

FIG. 10 is a schematic view of a fluid heating pump assembly in accordance with an embodiment of the present disclosure;

FIG. 11 is a schematic view of a fluid heating pump assembly in accordance with an embodiment of the present disclosure;

FIG. 12 is a schematic diagram of a fluid heat transfer system in accordance with an embodiment of the present disclosure;

FIG. 13 is a schematic diagram of a fluid heat transfer system in accordance with an embodiment of the present disclosure;

FIG. 14 is a schematic diagram of a fluid heat transfer system in accordance with an embodiment of the present disclosure;

FIG. 15 is a schematic view of a fluid heating pump assembly in accordance with an embodiment of the present disclosure; and

FIG. 16 is a schematic view of a fluid heating pump assembly in accordance with an embodiment of the present disclosure.

Referring to the drawings in which like reference characters refer to like parts throughout the several views thereof, the present disclosure is generally directed to an extracorporeal circulation system and a fluid heating pump assembly for use in such a system.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. Spatial or directional terms, such as “left”, “right”, “inner”, “outer”, “above”, “below”, and the like, are not to be considered as limiting as the disclosed embodiments can assume various alternative orientations.

As used herein, the singular form of “a”, “an”, and “the” include plural referents unless the context clearly dictates otherwise.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply examples of the disclosure. Hence, specific dimensions and other physical characteristics related to the examples disclosed herein are not to be considered as limiting.

The terms “first”, “second”, and the like are not intended to refer to any particular order or chronology, but refer to different conditions, properties, or elements.

The term “at least” is synonymous with “greater than or equal to”. The term “not greater than” is synonymous with “less than or equal to”.

It is to be understood that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the examples disclosed herein are not to be considered as limiting.

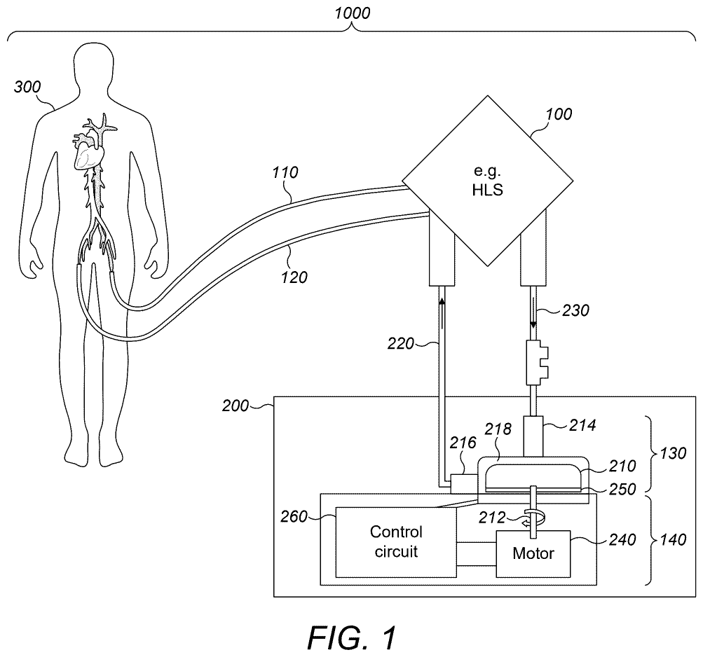

Referring first to FIG. 1, an extracorporeal circulation system 1000, (which may include or be referred to as a heart-lung machine, or as a cardiac bypass system, or as a cardiopulmonary bypass system, and which should be construed broadly to include cardiopulmonary bypass (CPB) systems, minimal extracorporeal circulation (MECC) systems, extracorporeal membrane oxygenation (ECMO) systems (respiratory and cardiac), and pump assisted lung protection (PALP) systems) is illustrated including an oxygenator/heat exchanger 100 and fluid heating pump assembly 200. The oxygenator/heat exchanger 100 is connected to a patient 300 via an inlet blood line 110 connected to a vein of the patient 300, and an outlet blood line 120 connected to an artery (or in some cases a vein) of the patient 300. The oxygenator/heat exchanger 100 may include an integrated heat exchanger, as illustrated in FIG. 1, or may be connected to an external heat exchanger. In other embodiments, no heat exchanger may be associated with the oxygenator. During an extracorporeal circulation procedure (e.g. an ECMO procedure), the oxygenator/heat exchanger 100 receives deoxygenated blood from the patient 300 via the inlet blood line 110, oxygenates the blood, and returns the oxygenated blood to the patient 300 via the outlet blood line 120. The oxygenator 100 may include or be connected to a blood circulation pump (not shown in FIG. 1) which circulates the blood through the inlet blood line 110, the oxygenator/heat exchanger 100, and the outlet blood line 120. Commercial examples of suitable blood circulation pumps and associated components include, but are not limited to, Maquet Cardiohelp® and Maquet Rotaflow® systems offered by Maquet Cardiopulmonary GmbH. The oxygenator/heat exchanger 100 and associated components of the system 1000 thus replicates the functionality of the patient's heart and/or lungs during an ECMO (or other extracorporeal circulation) procedure. The extracorporeal circulation system 1000 illustrated by FIG. 1 constitutes a simplified, non-limiting illustration, as such systems are generally much more complex.

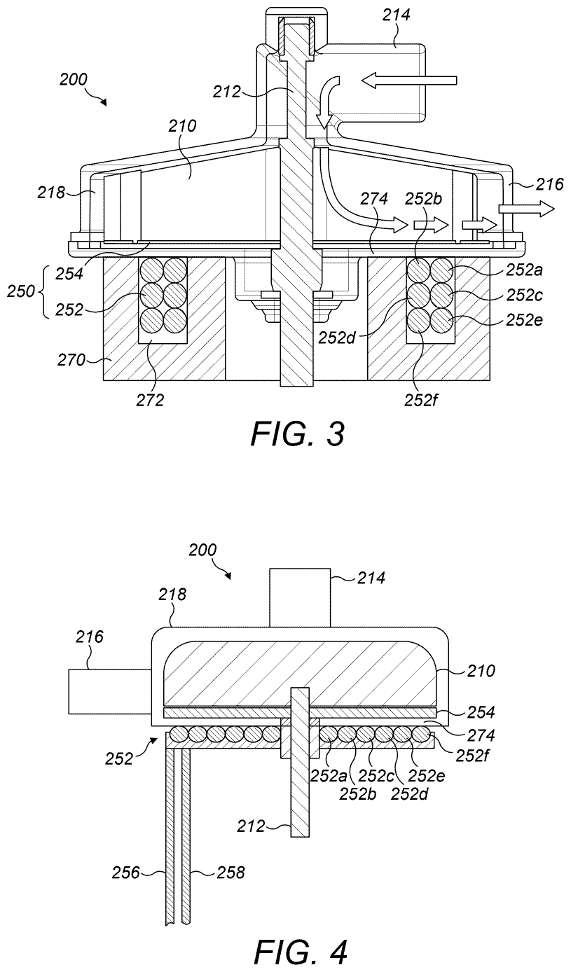

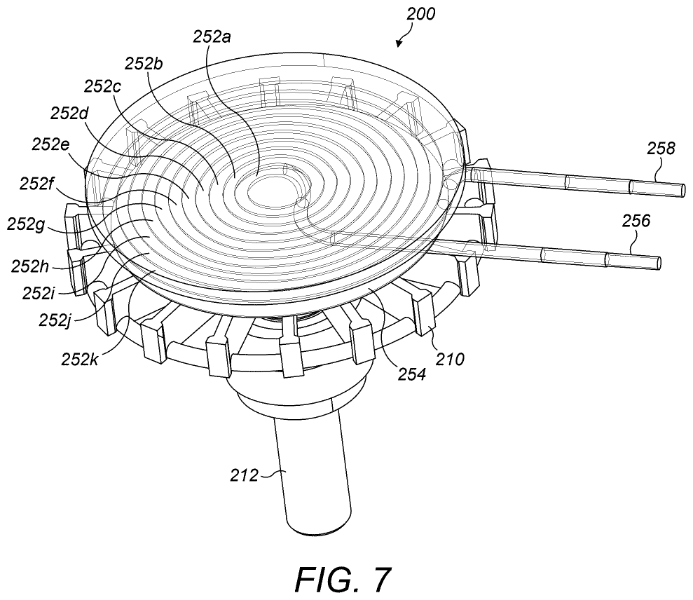

With continued reference to FIG. 1, the oxygenator/heat exchager 100 may include a heat exchanger portion configured to warm and/or cool the patient's blood to sustain a clinically desired blood temperature. To warm the patient's blood, the fluid heating pump assembly 200 supplies a heated warming fluid to the oxygenator/heat exchanger 100. The heated warming fluid directly or indirectly heats the blood in a heat exchanger portion of the oxygenator/heat exchanger 100, and is returned to the fluid heating pump assembly 200. The fluid heating pump assembly 200 includes an impeller 210, an outlet fluid line 220 through which the impeller 210 supplies heated warming fluid to the heat exchanger portion of the oxygenator/heat exchanger 100, and a return fluid line 230 through which the warming fluid is returned to the impeller 210 from the heat exchanger portion of the oxygenator 100. The fluid heating pump assembly 200 further includes a motor 240 which rotationally drives the impeller 210, a heating element 250 which heats the warming fluid, and an electronic controller 260. The electronic controller 260 may include, for example, circuitry for driving the motor 240 and for regulating the heating element 250 to control the temperature of the warming fluid. Further details of the fluid heating pump assembly 200 will be provided herein with reference to FIGS. 3-9.

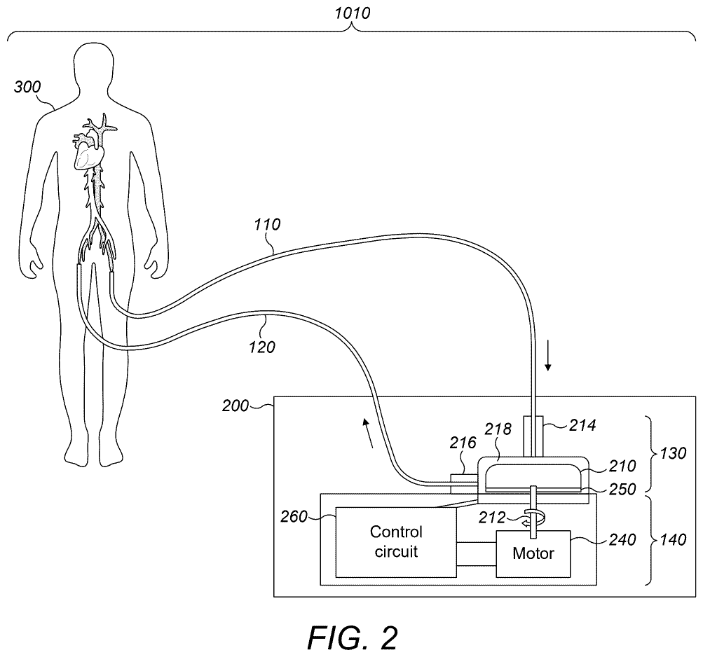

Referring now to FIG. 2, another embodiment of an extracorporeal circulation system 1010 is illustrated. Unlike the system 1000 of FIG. 1, the fluid heating pump assembly 200 of the system 1010 of FIG. 2 is connected directly to the patient 300, and the fluid heating pump assembly 200 circulates the blood to and from the patient 300. In particular, the inlet blood line 110 and the outlet blood line 120 are respectively connected to an inlet port 214 and an outlet port 216 of the fluid heating pump assembly 200. Thus, the fluid heating pump assembly 200 directly heats the patient's blood, without using a separate heat exchanger. As would be understood by those skilled in the art, various other system arrangements are possible, including an external heat exhanger, integrated heat exchanger/oxygenator, etc.

Referring FIGS. 1-2 embodiments of the fluid heating pump assembly 200 consists of a first component 130, the pump head, which includes an impeller chamber 218 in which the impeller 210 is disposed and of a second component 140 comprising the drive of the impeller. The first component is detachable from the second component. The impeller chamber 218 defines an inlet port 214 which receives warming fluid from the oxygenator/heatexchanger 100, and an outlet port 216 through which the warming fluid, after being heated, is returned back to the oxygenator/heatexchanger 100. The impeller 210 is coupled to an impeller shaft 212 which is rotated by the motor 240 (see FIGS. 1 and 2). The impeller shaft is typically part of the first component 130. In some embodiments the impeller shaft 212 may be part of the second component 140.

Referring now to FIGS. 3-7, and FIG. 16, the fluid heating pump assembly 200 further includes an inductive element being part of the second component 140, e.g. an inductor coil 252, and a conductor 254 being part of the first component 130 which together form the heating element 250 (FIG. 3). The conductor 254 is disposed within the first component 130 of the fluid heating pump assembly 200 such that the warming fluid circulated by the impeller 210 contacts the conductor 254 before exiting an outlet port 216 of the impeller chamber 218.

The inductor coil 252 receives electrical current from the controller 260 (see FIGS. 1 and 2) and generates an alternating magnetic field in response to the received electrical current. The magnetic field generated by the inductor coil 252 induces eddy currents in the conductor 254, which heat (i.e. raise the temperature of) the conductor 254. As illustrated in FIG. 3, the conductor 254 may be attached to the impeller 210, such that the warming fluid contacts the conductor 254 as the warming fluid is circulated by the impeller 210. As a result of the contact between the warming fluid and the conductor 254, the warming fluid is heated.

Heating efficiency is attributable to both macro-trubulence and micro-turbulence. As used herein, “macro-turbulence” refers to turbulence that can be seen with the naked eye. “Micro-turbulence”, by contrast, is used herein to refer to turbulence that occurs at a molecular level and thus cannot be seen directly by eye. However, micro-turbulence may be characterized by measuring various properties of fluid flow. Macro-turbulence of the warming fluid within the impeller chamber 218 may cause the warming fluid to mix with itself, resulting in a relatively even heat distribution within the warming fluid. As a result, the temperature throughout the warming fluid may be substantially consistent by the time the warming fluid exits the outlet port 216, without prominent hot spots and/or cold spots in the warming fluid. This ensures more reliable and predictable heating of the patient's blood within the oxygenator 100. Additionally, micro-turbulence directly surrounding the conductor 254 helps to minimize stagnant zones in the fluid to increase efficiency of heat transfer. Thus, the conductor 254 is a source of heat and also the source of microturbulence. This serves as an inherent safeguard, as halting rotation of the impeller 210 reduces microturbulence, which in turn reduces efficiency of the heat transfer through the fluid. Both macro-and micro-turbulence reduce the prevalence of localized heating of the fluid, and increase uniformity in heating the fluid.

As described above and with continued reference to FIG. 3, the inductor coil 252 is fluidly isolated from the impeller chamber 218 such that the warming fluid does not contact the inductor coil 252. For example, the inductor coil 252 may be fluidly isolated from the conductor 254 by a wall 274 forming a portion of the base 270 and/or the impeller chamber 218. The magnetic field generated by the inductor coil 252 is able to penetrate the wall 274 in order to induce eddy currents in the conductor 254, thereby heating the conductor 254 as described herein.

With continued reference to FIG. 3, in some embodiments, the inductor coil 252 may be disposed in a recess 272 in a base 270 of the fluid heating pump assembly 200. The base 270 may be made from a ferromagnetic material to increase inductive heating efficiency and form the electromagnetic field.

With continued reference to FIG. 3, the inductor coil 252 may include a continuous wire wound to form one or more windings 252a, 252b, 252c, 252d, 252e, 252f arranged concentrically about the impeller shaft 212. The number and arrangement of windings 252a, 252b, 252c, 252d, 252e, 252f can vary and determines the strength, direction, and associated characteristics of the magnetic field induced in the inductor coil 252 when electrical power is supplied to the inductor coil 252 by the controller 260. These characteristics of the magnetic field, as well as the proximity of the inductor coil 252 to the conductor 254, effect the heating of the conductor 254, and, consequently, heating of the warming fluid. In particular, electromagnetic coupling between the inductor coil 252 and the conductor 254 needs to be established in order to transfer energy.

In the embodiment illustrated in FIG. 3, the windings 252a, 252b, 252c, 252d, 252e, 252f of the inductor coil 252 are arranged in a multi-layer spiral fashion, with windings 252a and 252b forming a first layer nearest the conductor 254, windings 252c and 252d forming a middle layer, and winding 252e and 252f forming a third layer farther from the conductor 254.

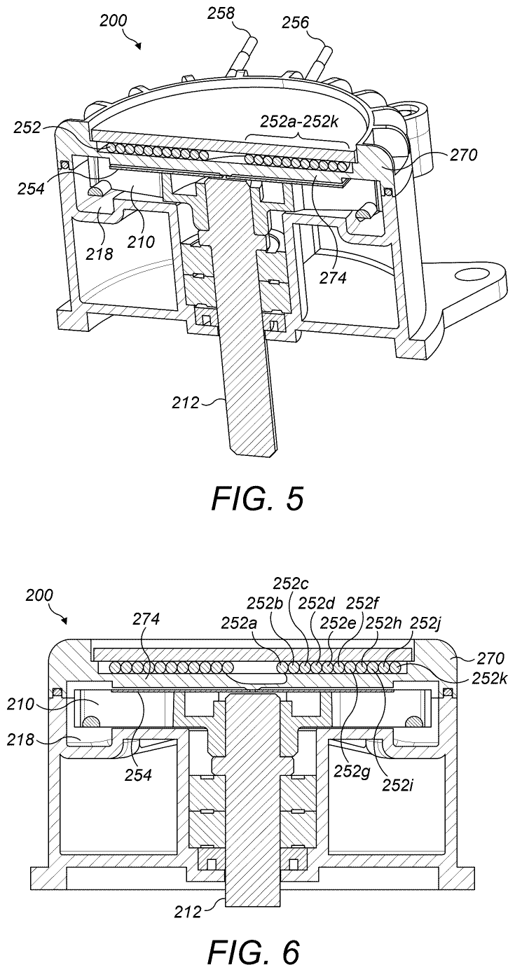

FIG. 4 illustrates another arrangement of the inductor coil 252 in which the windings 252a, 252b, 252c, 252d, 252e, 252f are all arranged in a single-layer spiral fashion. FIGS. 5-7 depict a similar embodiment to FIG. 4, but include additional windings 252g, 252h, 252i, 252j, 252k in the single-layer of windings. As also shown in FIGS. 4, 5, and 7, first and second leads 256, 258 extend from respective ends of the wire forming the inductor coil 252, with the leads 256, 258 being attached to the controller 260 to form an electrical circuit with the controller 260. For example, the first lead 256 may extend from the innermost winding 252a, and the second lead 258 may extend from the outermost winding 252k.

Though specific winding arrangments of the inductor coil 252 are shown in FIGS. 3-7, the present disclosure is not intended to be limited to such winding arrangments. Rather, the arrangement of the windings of the inductor coil 252 may take any form suitable to generate a sufficient electromagnetic field in the inductor coil 252 to produce the requisite amount of inductance to heat the conductor 254. Various changes may be made to the arrangement of windings to achieve the desired inductance. For example, the use of ferrite surrounding the windings may increase the generated inductance, thereby reducing the number of windings needed.

In some embodiments, the inductor coil 252 may be a provided as a printed circuit board (i.e. a PCB coil). Further, the inductor coil 252 is but one example of an inductive element that can be used to heat the conductor 254 via induction. In other embodiments, the inductor coil 252 may be substitiued for another inductive component for generating eddy currents in the conductor 254.

The inductor coil 252 may be a non-disposable component, that is not replaced along with the pump head. In some embodiments, the inductive coil 252 may be substititued for an alternative component having sufficient inductive properties to transfer energy to the conductor 254.

The conductor 254 is made from a material that exhibits an increase in temperature in the presence of the magnetic field generated by the inductor coil 252. That is, the conductor 254 is made from a material capable of being heated by induction. For example, the conductor 254 may be made from a ferrous metal, such as steel. As shown in FIGS. 3-7, the conductor 254 may include a substantially flat plate attached to the impeller 210 on a plane substantially perpendicular to the impeller shaft 212. In other embodiments, the conductor 254 may be embedded in the impeller 210, may be a conductive layer on the impeller or the impeller 210 may itself serve as the conductor 254. The conductor 254 may be a disposable component that is disposed of with the remainder of the head of the pump assembly 200.

Referring now to FIG. 16, another embodiment of the fluid heating pump assembly 200 is similar to the embodiments of FIGS. 3-7, but a conductor 259 is affixed within the impeller chamber 218 rather than being coupled to the impeller 210. Thus, the conductor 259 of the embodiment of FIG. 16 is stationary. The conductor 259 may consist of one single part placed at the botton, the side walls or the top of the impeller chamber or it may consist of several parts distributed in the impeller chamber. In some embodiments, the conductor 259 may be a conductive layer of the impeller chamber. The conductor 259 increases in temperature in response to electrical current being provided to the inductor coil 252 in the same manner as the conductor 254 of the embodiment of FIGS. 3-7. However, the embodiment of FIG. 16 may produce less micro-turbulence in the fluid being that the conductor 259 is stationary as opposed to rotating. In some embodiments the arrangement of the conductor may be in such a way that both conductor tpyes, a conductor 254 and a conductor 259, are implemented at the same time.

Referring now to FIG. 9, in another embodiment of the fluid heating pump assembly 200, the heating element 250 includes a conductor 255 rotationally coupled to the impeller 210. The conductor 255 includes a lug 266 that extends into an annular slot 262 of the impeller chamber 218. The conductor 255, including the lug 266, are made from a material capable of being heated via induction, such as a ferrous metal (e.g. steel). The annular slot 262 is partially surrounded by a permanent magnet 290 disposed outside the impeller chamber 218. The permanent magnet 290 may be substantially U-shaped or horseshoe-shaped such that the lug 266 of the conductor 255 extends between opposing sidewalls 292 of the permanent magnet 290. The permanent magnet 290 exhibits a magnetic field which induces eddy currents in the lug 266 as the lug 266 rotates through the magnetic field. Thus, rotation of the impeller 210, to which the lug 266 is rotationally coupled, causes the lug 266 to increase in temperature. Heat transfers to the remainder of the conductor 255 from the lug 266, and the warming fluid circulating in the impeller chamber 218 increases in temperature as the warming fluid contacts the conductor 255.

In some embodiments, the permanent magnet 290 can be moved towards and away from the conductor 255 in the direction D (see FIG. 9), parallel to the axis of rotation of the impeller 210, to vary the effect of the magnetic field on the lug 266. Moving the permanent magnet 290 toward the conductor 255 generally increases the magnitude of the eddy currents in the lug 266, thereby increasing the temperature of the conductor 255. Conversely, moving the permanent magnet 290 away from the conductor 255 generally decreases the magnitude of the eddy currents in the lug 266, thereby decreasing the temperature of the conductor 255. As the permanent magnet 290 exhibits a magnetic field in the absence of an external power source, heating of the conductor 255 and, consequently, the warming fluid is achieved using only the electromagnetic field passively generated by rotation of the lug 266 relative to the permanent magnet 290. Consequently, heat transfer to a fluid damageable by overheating (e.g. blood) could be stopped by stopping the rotation of the impeller. A similar embodiment to FIG. 9 may replace the permanent magnet 290 with an electromagnet, which would require an external power source to generate a magnetic field. The permanent magnet 290 (or the electromagnet) may include a single magnet or a plurality of smaller magnets.

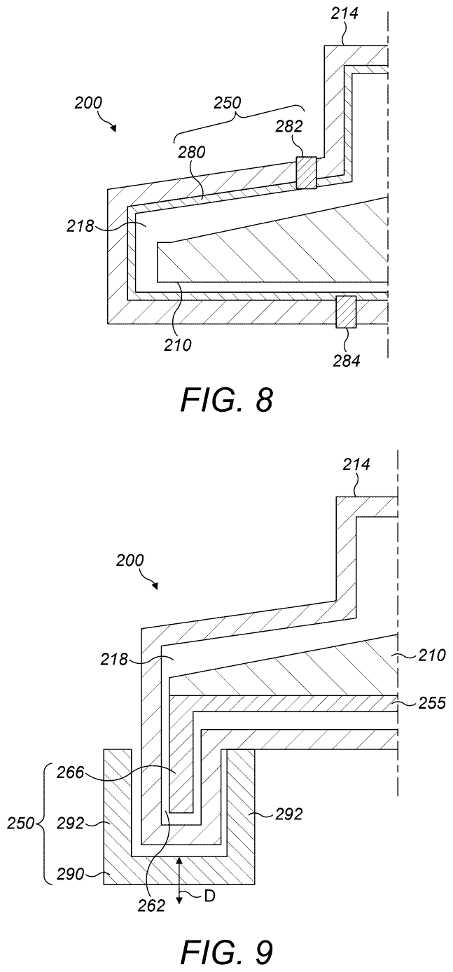

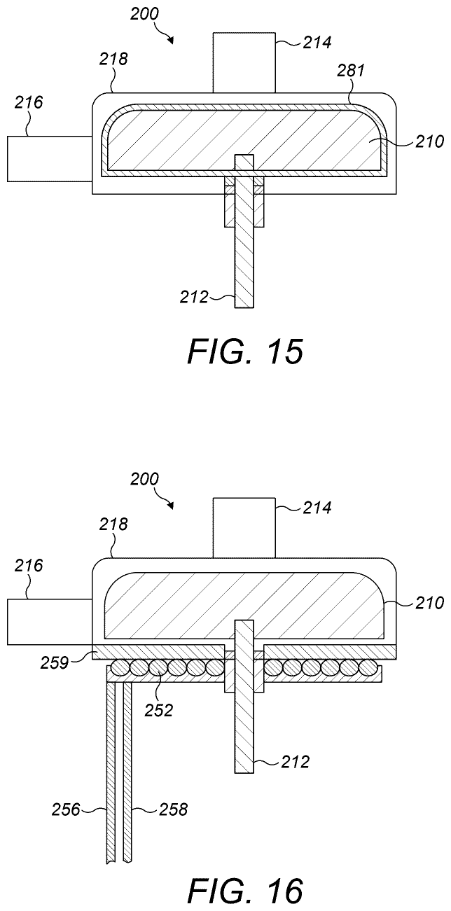

Referring now to FIGS. 8 and 15 other embodiments of the fluid heating pump assembly 200 utilize resitive heating to increase the temperature of the fluid in the impeller chamber 218. In the embodiment shown in FIG. 8, the heating element 250 includes an electrically conductive layer 280 applied to an inner surface of the impeller chamber 218. The conductive layer 280 includes at least two contacts 282, 284 that are connected to the controller 260 (see FIG. 1) in order to form an electrical circuit through which the controller 260 delivers electrical current to the conductive layer 280 via the contacts 282, 284. The conductive layer 280 increases in temperature in response to the electrical current from the controller 260. Consequently, the warming fluid circulating within the impeller chamber 218 is heated by coming into contact with the conductive layer 280. The electrical resistance of the conductive layer 280 and the electrical power supplied by the controller 260 determines the heating characteristics of the conductive layer 280. Thus, the conductive layer 280 may be selected to have a predetermined resistivity that achieves desired heating of the warming fluid in the impeller chamber 218. In some embodiments, the conductive layer 280 may be a coating applied to the impeller chamber 218, for example by a vapor deposition process.

In the embodiment shown in FIG. 15, the impeller 210 includes a conductive layer 281 (or at least a portion of the impeller 210 is itself conductive). The conductive layer 281 is electrically connected to the impeller shaft 212 so that the conductive layer 281 can receive electrical current from the electronic controller 260 (see FIG. 1) via the shaft 212, e.g. via a sliding contact of the shaft to the electronic controller. The conductive layer 281 increases in temperature in response to the electrical current from the controller 260. Consequently, the warming fluid circulating within the impeller chamber 218 is heated by coming into contact with the impeller 210. Heating of the impeller 210 may also induce micor-turbulence into the fluid at the surface of the impeller 210 which improves the efficiency and uniformity of heat transfer to the fluid. The electrical resistance of the conductive layer 281 and the electrical power supplied by the controller 260 determines the heating characteristics of the conductive layer 281. Thus, the conductive layer 281 may be selected to have a predetermined resistivity that achieves desired heating of the warming fluid in the impeller chamber 218. In some embodiments, the conductive layer 281 may be a coating applied to the impeller 210, for example by a vapor deposition process.

In some embodiments the conductive parts 280 and 281 and their contacts to power may be combined in the same pump head.

As may be appreciated from the present disclosure inclusive of the accompanying drawings, the pump assembly 200 may take various forms for effecting heat transfer to the fluid in the impeller chamber 218. The embodiments of the pump assembly 200 shown in FIGS. 3-7 and 9-11 utilize inductive heating to heat a conductor 254, 255 attached (e.g. rotationally coupled) to the impeller 210. In contrast, the embodiments of the pump assembly 200 shown in FIGS. 8 and 15 utilize resistive heating to heat a conductive layer 280, 281 on the impeller chamber 218 and/or the impeller 210. Thus, embodiments of the present disclosure may be distinguished by the method of heating (inductive versus resistive) used to transfer heat to the fluid. In some embodiments, both forms of heating may be combined, for example, by using the conductor 254 of FIGS. 3-7 along with the conductive layer 280 of FIG. 8.

Furthermore, heating may be achieved by heating either or both of the impeller 210 and the impeller chamber 218. In the embodiments shown in FIGS. 3-7, 9-11, and 15 heating occurs on the impeller 210, and thus the heating element is rotating. In contrast, in the embodiments of FIGS. 8 and 16, the heating occurs at the impeller housing 218, and thus the heating element is stationary. In some embodiments, the pump assembly 200 may incorporate both rotating and stationary heating elements, such as by combining the inductive impeller heating of FIGS. 3-7 with the resistive impeller chamber heating of FIG. 8.

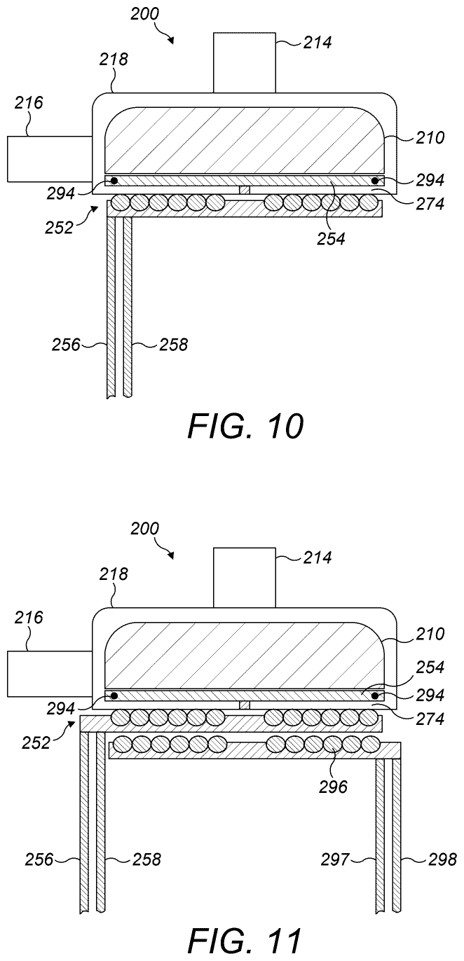

Referring now to FIGS. 10 and 11, alternate embodiments of the fluid heating pump assembly 200 are schematically illustrated. The fluid heating pump assembly 200 of FIGS. 10 and 11 may be similar to that of FIG. 4, except that instead of the impeller 210 being connected to a motor-driven shaft, the impeller 210 is rotationally driven by an electromagnetic drive. The impeller 210 includes one or more magnets 294 (e.g. permanent magnets) arranged an operative distance from an electromagnetic drive located outside the impeller chamber 218. The electromagnetic drive interacts with the magnets 294 to rotate the impeller 210 via induction. As such, the impeller 210 need not be coupled to a shaft extending outside of the impeller chamber 218 to a motor. Instead, the impeller 210 may either be mechanically supported within the impeller chamber 218 (e.g. by one or more bearings), or the impeller 210 may be magnetically suspended (i.e. levitated) within the impeller chamber 218 for a friction-free connection. In some embodiments, as shown in FIG. 10, the inductor coil 252 itself also serves as the electromagnetic drive. Thus, the electrical current supplied to the inductor coil 252 both heats the conductor 254 and rotates the impeller 210. In other embodiments, as shown in FIG. 11, a separate drive coil 296 may be provided outside the impeller chamber 218 to serve as the electromagnetic drive for inductively rotating the impeller 210. The drive coil 296 may include leads 297, 298 connected to the electronic controller 260 (see FIG. 1) for supplying power to the drive coil 296. The drive coil 296 may include one or more windings arranged in various arrangements (e.g. one or more layers) to achieve the required and/or desired level of electromagnetic induction to drive the impeller. In other embodiments, the electromagnetic drive may be a source of electromagnetic induction other than a coil.

While embodiments of the present disclosure have generally been described in connection with an ECMO system, the fluid heating pump assembly 200 and associated components described herein can be utilized in a variety of extracorporeal blood flow systems including ECMO, a heart-lung machine, a cardiopulmonary bypass machine, and a pump-assisted lung protection machine. Furthermore, the fluid heating pump assembly 200 is not limited to heating a warming fluid, and may instead be used to heat and/or cool any non-bioilogical fluid or biological fluid (e.g. blood in an ECLS procedure). The fluid heating pump assembly 200 may also be used outside the field of extracorporeal circulation, to heat and pump fluids in a variety of other applications where efficient heating of the fluid is desired in a compact device. Embodiments of the pump assembly 200 described in the present disclosure also maintain sterility of the circulated fluid during the heating and/or cooling process.

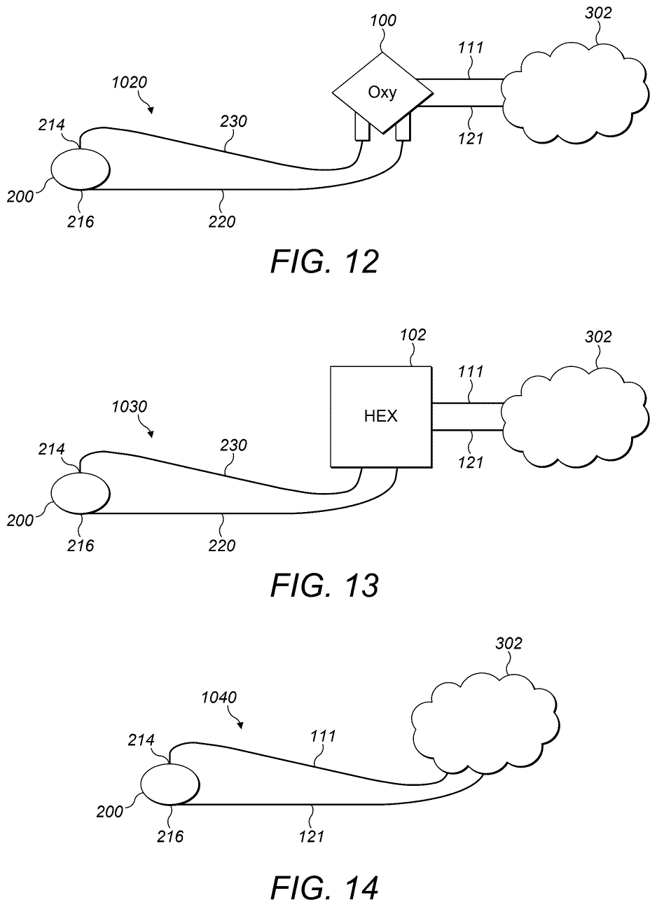

FIGS. 12-14 illustrate schematics for other examples of circulation systems according to the present disclosure. The systems shown in FIGS. 12-14 may include many of the same components previously described with reference to FIGS. 1-11, and like reference numerals in the figures refer to like components. FIG. 12 shows a system 1020 inlcuding an integrated oxygenator/heat exchanger 100 in connection with a volume of fluid 302 to be heated. The volume of fluid 302 may be any biological or non-biological fluid to be circulated through the oxygenator/heat exchanger 100. The volume of fluid 302 is fluidly connected to the oxygenator/heat exchanger 100 via an inlet fluid line 111, through which the fluid enters the oxygenator/heat exchanger 100, and an outlet fluid line 121, through which the fluid returns to the volume of fluid 302. The oxygenator/heat exchanger 100 is in turn connected to the inlet port 214 and outlet port 216 of the fluid heating pump assembly 200 via the the return fluid line 230 and the outlet fluid line 220, respectively. The system 1020 of FIG. 12 is thus a genericized embodiment of the system 1000 of FIG. 1, and can be utilized in any fluid circuit heating a biological or non-biological fluid. In some examples, the volume of fluid 302 of the system 1020 of FIG. 12 may correspond to the patient 300 of the system 1000 of FIG. 1, with the circulated fluid being the patient's blood.

FIG. 13 shows a system 1030 including a heat exchanger 102 in place of the oxygenator/heat exchanger 100 of FIG. 12. The heat exchanger 102 is not integrated with an oxygenator, so oxygenation of the fluid, if desired, is performed in a separate component. In some examples, the volume of fluid 302 of the system 1030 of FIG. 13 may correspond to the patient 300 of the system 1000 of FIG. 1, with the circulated fluid being the patient's blood.

FIG. 14 shows a system 1040 lacking both a heating exchanger and oxygenator. The volume of fluid 302 is circulated directly through the fluid heating pump assembly 200, without utilizing an intervening warming fluid circuit. In particular, the inlet fluid line 111 and the outlet fluid line 121 are respectively connected to the inlet port 214 and the outlet port 216 of the fluid heating pump assembly 200. Thus, the fluid heating pump assembly 200 directly heats the fluid from the volume of fluid 302, without using a separate heat exchanger. The system 1040 of FIG. 14 is a generalized embodiment of the Heart Assist system 1010 of FIG. 2, and can be utilized in any fluid circuit heating a biological or non-biological fluid. In some examples, the volume of fluid 302 of the system 1040 of FIG. 14 may correspond to the patient 300 of the system 1010 of FIG. 2, with the circulated fluid being the patient's blood. As the heating circulation pump transfers heat only when running heat transfer to a fluid damageable by overheating (e.g. blood) could be stopped by stopping the pump.

While various examples of the present disclosure were provided in the foregoing description, those skilled in the art may make modifications and alterations to these examples without departing from the scope and spirit of the disclosure. For example, it is to be understood that features of various embodiments described herein may be adapted to other embodiments described herein. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

We claim:

1. A heating fluid pump assembly for a fluid flow system, the heating fluid pump assembly comprising:
a first component comprising an impeller configured to circulate a fluid through an impeller chamber;
a second component comprising the drive of the impeller; and
an inductive heating element, the inductive heating element comprising a conductor which is part of the first component, and an inductor which is part of second component, fluidly isolated from the conductor,
wherein the first component is detachable from the second component,
wherein the inductor is configured to generate a magnetic field to induce eddy currents in the conductor,
wherein the heating element is configured to induce eddy currents in the conductor in order to heat the conductor, and
wherein the conductor is configured to contact the fluid as the fluid is circulated by the impeller.

2. The heating fluid pump assembly of claim 1, wherein the inductive heating element comprises an inductor coil.

3. The heating fluid pump assembly of claim 2, further comprising a controller configured to supply electrical power to the inductor coil to generate the magnetic field.

4. The heating fluid pump assembly of claim 2, wherein the inductor coil comprises one or more windings.

5. The heating fluid pump assembly of claim 4, wherein the one or more windings are arranged in a single layer.

6. The heating fluid pump assembly of claim 4, wherein the one or more windings are arranged in multiple layers.

7. The heating fluid pump assembly of claim 4, wherein the inductor coil is a printed circuit board.

8. The heating fluid pump assembly of claim 1, wherein the conductor is rotationally coupled to the impeller.

9. The heating fluid pump assembly of claim 1, wherein the impeller as a whole is made of conductive material so that the impeller is the conductor at the same time.

10. The heating fluid pump assembly of claim 9, wherein the conductive impeller includes an arrangement to increase the surface resulting in an increased heat exchange area and induces micro-turbulence in the fluid.

11. The heating fluid pump assembly of claim 1, wherein the conductor is configured as a conductive layer applied to an inner surface of the impeller chamber.

12. The heating fluid pump assembly of claim 11, further comprising a controller configured to supply electrical power to the conductive layer.

13. The heating fluid pump assembly of claim 1, wherein the conductor is configured as a stationary heating component affixed to the impeller chamber.

14. The heating fluid pump assembly for a fluid flow system of claim 1,
comprising at least one of an ECLS machine, a heart-lung machine, and a dialysis machine,
wherein the circulating fluid is blood, and
wherein the blood is circulated and heated in a circuit comprising the extracorporeal part of the circuit, the circulatory system of the patient and at least one inflow and one outflow arrangement connecting the extracorporeal part of the circuit to the circulatory system of the patient,
wherein the extracorporeal part of the circuit comprises the first component comprising an impeller and an impeller chamber and conduits connecting the first component to the inflow and outflow arrangement.

15. A heating fluid pump assembly for a fluid flow system, the heating fluid pump assembly comprising:
a first component comprising an impeller configured to circulate a fluid through an impeller chamber;
a second component comprising the drive of the impeller; and
an inductive heating element, the inductive heating element comprising a conductor which is part of the first component, and an inductor which is part of second component, fluidly isolated from the conductor,
wherein the first component is detachable from the second component,
wherein the inductor is configured to generate a magnetic field to induce eddy currents in the conductor,
wherein the heating element is configured to induce eddy currents in the conductor in order to heat the conductor,
wherein the conductor is configured to contact the fluid as the fluid is circulated by the impeller; and
wherein the first component comprising an impeller and an impeller chamber is connected via tubes to a heat exchanger to constitute a warming fluid circuit and wherein the warming fluid circuit is configured to circulate a warming fluid through the impeller chamber and through the heat exchanger.

16. The heating fluid pump assembly for a fluid flow system of claim 15 wherein the warming fluid circuit is prefilled with a sterile warming fluid and wherein the sterile warming fluid circulates through the heat exchanger.

17. The heating fluid pump assembly for a fluid flow system of claim 15 for ECMO applications,
wherein an initial warming fluid circuit comprising of the said first component comprising an impeller and an impeller chamber and of a conduit connecting input and output of the impeller chamber is prefilled with a sterile warming fluid and
wherein the initial warming fluid circuit is connected under sterile conditions to the heat exchanger integrated in an oxygenator so that during the ECMO case sterile warming fluid circulates through the heat exchanger.

18. The heating fluid pump assembly for a fluid flow system of claim 15 comprising:
at least one of an ECMO machine, a heart-lung machine, and a dialysis machine,
wherein the circulating fluid is blood, and
wherein the blood is circulated and heated in a circuit comprising the extracorporeal part of the circuit, the circulatory system of the patient and at least one inflow and one outflow arrangement connecting the extracorporeal part of the circuit to the circulatory system of the patient,
wherein the extracorporeal part of the circuit comprises the first component comprising an impeller and an impeller chamber, an oxygenator, and conduits connecting the first component to the oxygenator and to the inflow and outflow arrangement.

19. The heating fluid pump assembly of claim 15, wherein the inductive heating element comprises an inductor coil.

20. The heating fluid pump assembly of claim 15, further comprising a controller configured to supply electrical power to the inductor coil to generate the magnetic field.
